(19) United States
(12) Patent Application Publication
(10) Pub. No.: US 2006/0212099 A1
(43) Pub. Date: Sep. 21, 2006

(54) Optical skin germicidal device and method

(76) Inventor: Robert H. Riddell, Bellevue, WA (US)

Correspondence Address: DEAN A. CRAINE, STE 140, 400 - 112TH AVE NE, BELLEVUE, WA, 98004, US

(21) Appl. No.: 11/080,688

(22) Filed: Mar. 15, 2005

(51) Int. Cl. A61N 5/06

(52) U.S. Cl. 607/88; 607/94

(57) ABSTRACT

An optical skin germicidal device and method that uses germicidal ultraviolet light to treat local infections. The device includes a germicidal light source connected to a flexible optical fiber and a hollow needle designed to receive the optical fiber. The optical fiber, which is designed to slide freely inside the needle, is covered with cladding except for the distal end or section that slides into the needle. Formed on the inside surface of the needle is an elongated slot that allows rays of optical light to exit the needle. The slot is sufficient in width so that the optical fiber remains at all times inside the curved needle. By moving the optical fiber along the needle, the tissue surround the inside surface of the needle adjacent to the slot may be treated with germicidal light. Using the device, a method of treatment is also provided.

### BACKGROUND OF THE INVENTION

1. Field of the Invention

The invention disclosed herein pertains to sterilization devices and, more particularly to devices used to sterilize skin or living tissue using germicidal light.

2. Description of the Related Art

Antibiotic resistant bacteria is a major concern in medicine and dentistry. Safe, effective alternative methods for treating bacteria infections that do not rely on antibiotics or other chemicals are needed.

It is also well known that non-vascular areas in the human body with limited blood flow are difficult to treat with oral or IV or IM administered antibiotics. If the infection site is below the skin, typical antibiotics have limited penetration through the skin and are generally, ineffective.

Because ultraviolet light does not substantially penetrate living tissue, heretofore, it has not be used to treat infections. In addition, because ultraviolet light is widely known to cause optical damage, most medical and dental practitioners are reluctant to use it on their patients.

### SUMMARY OF THE INVENTION

It is an object of the present invention to provide a tool for safely and effective applying germicidal ultraviolet light to living tissue

It is another object to provide such a tool that allows the ultraviolet light to be applied relatively close to the infected issue thereby reducing damaging to the surrounding non-infective living tissue.

It is another object of the present invention to provide such a tool that is safe and easy to use.

It is a further object of the invention to provide a method of applying germicidal light to subcutaneous living tissue.

These and other objects of the present invention are met by the optical germicidal tool and method disclosed herein that uses a germicidal ultraviolet light source connected to a flexible optical fiber that slides freely inside the center bore formed in a rigid or semi-rigid hollow straight or curved needle designed to be inserted into living tissue. Formed on one side of the needle is at least one elongated slot that allows the rays of optical light transmitted from the distal end or distal section of the optical fiber to be transmitted into the tissue adjacent to the slot. The entire optical fiber is covered with protective cladding except for the distal section or distal end that fits into the needle. The elongated slot formed on the side of the needle is sufficiently narrow in width so that the optical fiber remains inside the needle at all times. During use, the operator moves the needle so that the elongated slot is positioned at a desired position adjacent to infected tissue. The needle is then rotated so that the elongated slot directly faces the infected tissue. The optical fiber is then rotated and longitudinally aligned inside the needle so that the distal section or distal end of the optional fiber is positioned adjacent to the slot. After the tissue has been treated, the needle may be rotated again inside the tissue so that other adjacent areas of infected tissue may be exposed to germicidal light.

Using the above-described tool, a method of treatment of an infected tissue with germicidal light is also provided.

### DESCRIPTION OF THE DRAWINGS

FIG. 1 is a side elevational view of the germicidal device disclose herein.

FIG. 2 is a sectional side elevational view of fiber optic with a bare distal section.

FIG. 3 is a sectional side elevational view of the fiber optic with the distal section covered with cladding and the distal end being cut at a critical angle to emit light at a critical angle.

FIG. 4 is a side elevational view of a fiber optic placed inside a straight needle with one elongated slot formed on its side.

FIG. 5 is a front sectional view of the straight needle embodiment of the invention showing the needle rotated in one direction to transmit germicidal light in one direction inside the tissue

FIG. 6 is a front sectional view of the straight needle embodiment shown in FIG. 5 with the needle rotated to transmit germicidal light in the opposite direction inside the tissue.

FIG. 7 is a side elevational view of the curved needle embodiment of the invention.

FIG. 8 is a perspective view of the exposed fiber optic being inserted into the proximal end of the handle.

### DESCRIPTION OF THE PREFERRED EMBODIMENT(S)

As shown in the accompanying Figs is an optical germicidal tool 10 that uses a germicidal ultraviolet source 12 connected to a flexible optical fiber 20 that is inserted into a hollow needle 30 that is then directly inserted into infected tissue. The optical fiber 20, which is made of flexible material, is designed to slide freely inside the center bore 32 formed in the needle 30. The optical fiber 20 is made of a single or a plurality of thin optical strands 24 bundled together and covered with a cladding 26. In the first embodiment, the distal section 27 of the optical fiber 20 is bare and designed to be inserted completely into the needle 30. In the second embodiment, the optical fiber, designated 20′, is covered completely with cladding 26 and bare only at its distal end 28. The distal end 28 may be sloped at a critical angle to allow rays of optical light to exit the distal end of the fiber 20′ and reflect internally inside the needle 30 and eventually through the elongated slot 50.

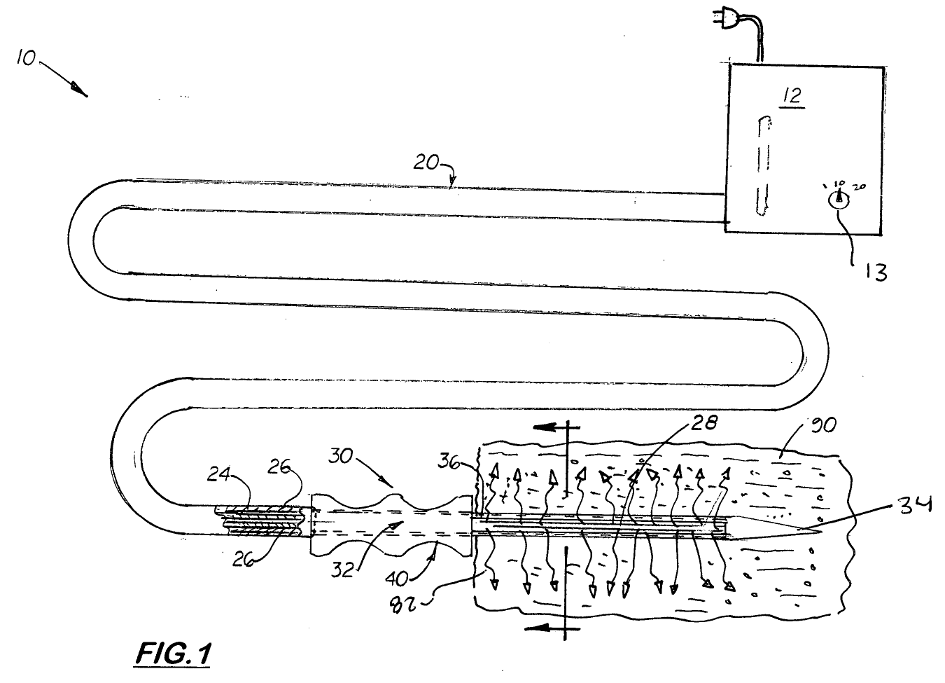

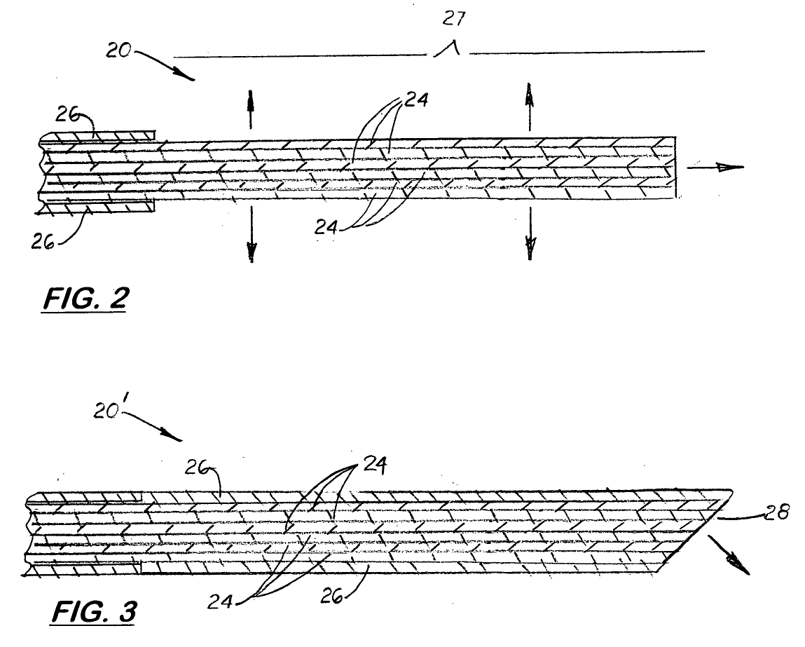

In the first embodiment shown in FIGS. 1 and 2, the needle 30 is a straight structure with a closed, sharp distal end 34 designed to penetrate living tissue 90. Attached to the proximal end 36 of the needle 30 is a large knob or handle 40 that the operator grabs to maneuver the needle 30 into and around the infected tissue 90. The proximal end 36 of the needle 30 extends slightly beyond the outer end of the handle 40 thereby allowing the optical fiber 20 to be easily inserted into the needle'center bore 32.

In a second embodiment, the tool 10 includes a curved needle, designated 30′, designed to be used to treat infected tissue 90 that cannot be accessed with the straight needle 30. The curve needle 30′ includes a closed sharp distal end 34′ and an optical handle 40′ formed at its proximal end 36′.

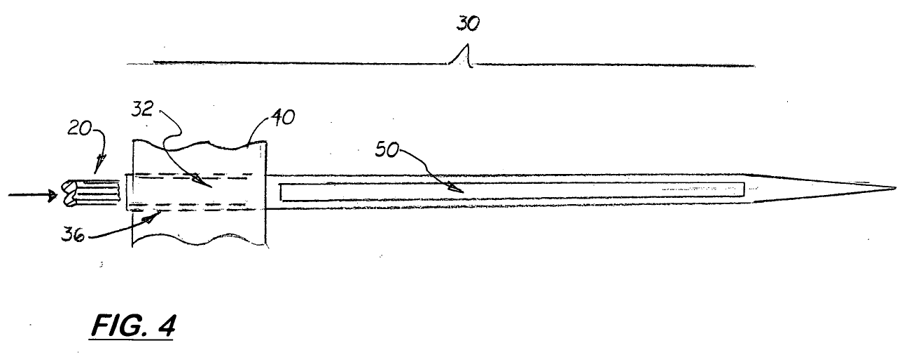

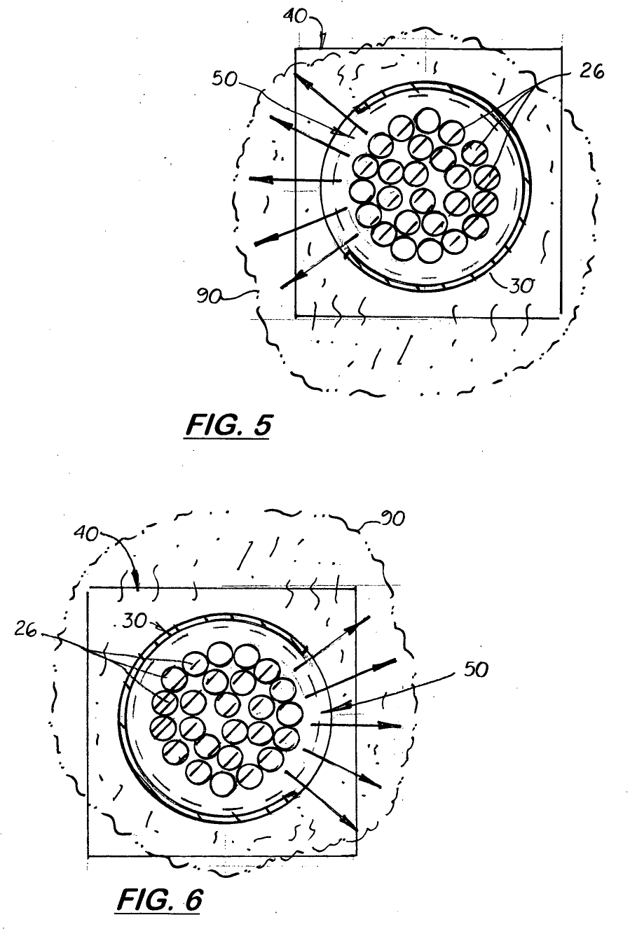

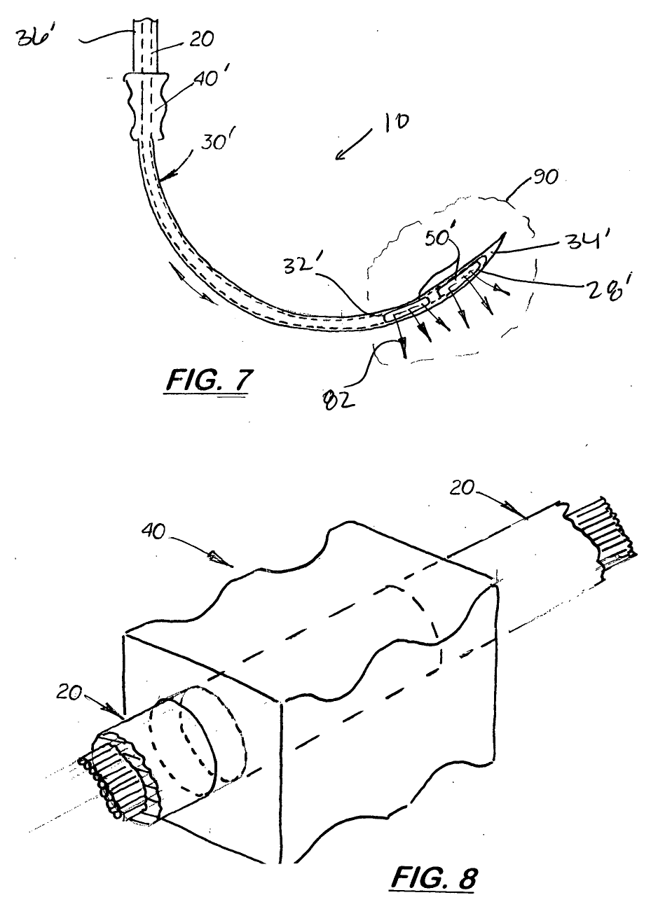

Formed on the surface of the needle 30, 30′ is at least one elongated slot 50 that allows rays of the germicidal light 82 from the distal section 27 or distal end 28 of the optical fiber 20, 20′ to exit the needle 30. The slot 50 may extend the entire length of the needle as shown in FIGS. 1 and 4 or be replaced with a plurality of longitudinally aligned, short slots 50′ as shown in FIG. 7. In both embodiments, the slot 50 or slots 50′ are sufficiently narrow so that the optical fiber 20 remains longitudinally aligned inside the needle 30 at all times. In the preferred embodiment, the optical fiber is approximately 1/16 inch in diameter and 36 to 48 inches in length. The needle 30 is a hollow surgical needle approximately 2 to 5 inches in length with a diameter gauge between 24 and 16. The center bore 32, 32′ of the needle 30, 30′ is sufficient in diameter so that the optical fiber 20 may slide freely therein. The radius of the curved needle 30′ is approximately 8 inches. It should be understood however, that other sizes of straight and curved needles 30, 30′, respectfully, may be used.

As mentioned above, attached to the proximal end 29 of the optical fiber 20 is a germicidal light source 12. In the preferred embodiment, the germicidal light source 12 is identical to the germicidal light 12 shown and described in the Applicant's earlier filed U.S. patent applications (Ser. No. 10/865,877, entitled Germicidal Toothbrush and Holder filed on Jun. 14, 2004; and Ser. No. 11,053,089, entitled Germicidal Brush Cleaner, filed on Feb. 7, 2005). The germicidal ultraviolet light source 12 is specifically used to produce coherent light at a wavelength of approximately 256 nm. In the preferred embodiment, the light source 12 is a portable, battery operated or AC powered and contains at least one UV lamp and includes an adjustable timer.

During use, the needle 30 or 30′ is placed adjacent to or inside the infected tissue 90 to be treated. Once properly positioned, the needle 30, 30′ is then selectively rotated so that the elongated slot 50 or slots 50′ are positioned against the tissue 90. The optical fiber 20 is then inserted into the needle 30, 30′ so that its distal section 27 or distal end 28 is adjacent to the elongated slot 50 or slots 50′. The timer 13 is then set for a 20 to 30 second time period. Once properly positioned and the timer 13 is set, the germicidal light source 12 is then activated. The timer 13 on the germicidal light source 12 is used to track the amount of time (20-30 seconds). Once the treatment is completed, the needle 30 is then rotated or repositioned inside the tissue 90 to treat another area. The timer 13 is then activated again to control the length of exposure.

The curved needle 30′ may be made of optical reflective material, such as stainless steel. When the optical fiber 20 is placed inside the curve needle 30′, the ultraviolet is transmitted from the distal end 28′ of the optical fiber 20 at a critical angle so that the ultraviolet light is reflected off the inside surfaces of the needle 30′ and through the slot 50 or slots 50′

According to Dr. Harry T. Whelan, Medical College of Wisconsin and NASA-Marshall Space Center, Huntsville Ala., ultra light combining wavelengths of light 680, 730, and 880 nm, each at 4 Joules cm squared (near infrared) can penetrate to a depth of 23 cm without damaging skin. It is postulated that germicidal light at 256 nm can have a germicidal effect to a depth of approximately 1 cm. Because the germicidal light may have a lethal effect on healthy body tissue at considerable depth, healthy body tissue must be protected from germicidal light. By using an elongated slot to conduct the transmission of light, healthy tissue is protected.

The following are examples of possible treatments that the device 10 may be used:

1. Exposing the 5^{th} nerve on the side of the face with germicidal light to stop Tic Dou-Lou-Reux;

2. Exposing the nerves to stop Herpes if there is an eruption of the virus on the outside of the body;

3. Exposing boils with germicidal light;

4. Exposing some cancers with germicidal light in an early stage like ‘Melanoma’;

5. Exposing ‘Flesh eating bacteria’ or ‘Necrotizing Faciitis’. These would use either designed attachment but in different sizes depending on the size that would be required;

6. Exposing spores at NASA's Jet Propulsion Lab so they don't contaminate the universe. A long optical fiber is all that would be necessary to reach into difficult areas;

7. Exposing large intestine polyps and Ulcers in the Stomach rather than surgery. These would require a micro instrument that could be small enough to be inserted through the large intestine or esophagus. A hood would be required to protect the gut or esophagus from injury on insertion and extraction, and a hood also to protect the adjacent healthy tissue from the germicidal light. Specifically trained surgeons would use micro instruments to be able to do this work.

In compliance with the statute, the invention described herein has been described in language more or less specific as to structural features. It should be understood, however, that the invention is not limited to the specific features shown, since the means and construction shown is comprised only of the preferred embodiments for putting the invention into effect. The invention is therefore claimed in any of its forms or modifications within the legitimate and valid scope of the amended claims, appropriately interpreted in accordance with the doctrine of equivalents.

1. An optical germicidal tool, comprising:
a. a germicidal light source;
b. a flexible optical fiber connected to said germicidal light source; and,
c. a hollow needle designed to receive the optical fiber, said needle including a center bore sufficient in diameter to allow said optical fiber to slide freely therein, said needle includes an inside surface with at least one elongate slot to allow germicidal light transmitted by said optical fiber to escape into surround tissue when said needle is inserted into said tissue.

2. The optical germicidal tool, as recited in claim 1, wherein said elongated slot extends substantially the entire length of said needle.

3. The optical germicidal tool, as recited in claim 2, wherein said elongated slot is sufficiently narrow to keep said optical fiber inside said needle.

4. The optical germicidal tool, as recited in claim 1, further including a handle attached to said needle.

5. The optical germicidal tool, as recited in claim 2, further including a handle attached to said needle.

6. The optical germicidal tool, as recited in claim 3, further including a handle attached to said needle.

7. The optical germicidal tool, as recited in claim 1, wherein said needle is straight.

8. The optical germicidal tool, as recited in claim 2, wherein said needle is straight.

9. The optional germicidal tool, as recited in claim 3, wherein said needle is straight.

10. The optional germicidal tool, as recited in claim 1, wherein said needle is straight.

11. The optional germicidal tool, as recited in claim 2, wherein said needle is straight.

12. A method for treating a localized infection is provided consisting of the following steps:
a. selecting a germicidal light source;
b. attaching an optical fiber to said germicidal light source, said optical fiber having an open distal end;
c. selecting a needle with a center bore sufficient in size to allow said optical fiber to slide freely therein, said needle including at least one side elongated slot;
d. positioning said needle adjacent to an infected tissue;
e. inserting said optical fiber into said needle so that said distal end of the optical fiber is positioned adjacent to said elongated slot so that germicidal light from the distal end of the optical fiber is transmitted into the infected tissue adjacent to said elongated slot.
